(19) United States
(12) Patent Application Publication
(10) Pub. No.: US 2014/0207136 A1
(43) Pub. Date: Jul. 24, 2014

(54) MULTIPLE STAGGERED ELECTRODES CONNECTED VIA FLEXIBLE JOINTS

(71) Applicant: St. Jude Medical, Inc., St. Paul, MN (US)

(72) Inventors: Alan DE LA RAMA, Cerritos, CA (US); Cary HATA, Irvine, CA (US)

(73) Assignee: St. Jude Medical, Inc., St. Paul, MN (US)

(21) Appl. No.: 13/886,332

(22) Filed: May 3, 2013

Related U.S. Application Data
(60) Provisional application No. 61/642,643, filed on May 4, 2012.

(51) Int. Cl. A61B 18/14

(52) U.S. Cl. CPC A61B 18/1492
USPC 606/41

(57) ABSTRACT

An ablation catheter includes an electrode assembly having a plurality of longitudinally spaced sets of spines, which include a distal set of spines each having a distal end connected to a spine distal junction and a proximal end connected to a first joint, and a proximal set of spines each having a proximal end connected to the distal end of the catheter body and a distal end connected to the first joint or a second joint. The spines include electrodes. Zero or more additional sets of spines are connected between the distal set and proximal set, such that neighboring sets of spines are connected by a joint. Each joint is flexible to permit bending along the longitudinal axis. The electrode assembly is movable between a collapsed arrangement and an expanded arrangement with the intermediate segments of the spines in the expanded arrangement moving outwardly with respect to the collapsed arrangement.

### RELATED APPLICATIONS

This application claims the benefit of U.S. Provisional Patent Application No. 61/642,643, file May 4, 2012, the entire disclosure of which is incorporated herein by reference.

### BACKGROUND OF THE INVENTION

The present invention relates generally to ablation devices and, more specifically, to an assembly of ablation elements such as electrodes arranged in a staggered configuration having multiple baskets/sets of spines of staggered ablation elements, wherein each pair of longitudinally neighboring baskets/sets of spines are separated by a flexible joint.

Hypertension is a major global public health concern. An estimated 30-40% of the adult population in the developed world suffers from this condition. Furthermore, its prevalence is expected to increase, especially in developing countries. Diagnosis and treatment of hypertension remain suboptimal, even in developed countries. Despite the availability of numerous safe and effective pharmacological therapies, including fixed-drug combinations, the percentage of patients achieving adequate blood-pressure control to guideline target values remains low. Much failure of the pharmacological strategy to attain adequate blood-pressure control is attributed to both physician inertia and patient non-compliance and non-adherence to a lifelong pharmacological therapy for a mainly asymptomatic disease. Thus, the development of new approaches for the management of hypertension is a priority. These considerations are especially relevant to patients with so-called resistant hypertension (i.e., those unable to achieve target blood-pressure values despite multiple drug therapies at the highest tolerated dose). Such patients are at high risk of major cardiovascular events.

Renal sympathetic efferent and afferent nerves, which lie within and immediately adjacent to the wall of the renal artery, are crucial for initiation and maintenance of systemic hypertension. Indeed, sympathetic nerve modulation as a therapeutic strategy in hypertension had been considered long before the advent of modern pharmacological therapies. Radical surgical methods for thoracic, abdominal, or pelvic sympathetic denervation had been successful in lowering blood pressure in patients with so-called malignant hypertension. However, these methods were associated with high perioperative morbidity and mortality and long-term complications, including bowel, bladder, and erectile dysfunction, in addition to severe postural hypotension. Renal denervation is the application of a chemical agent, or a surgical procedure, or the application of energy to partially or completely damage renal nerves to partially or completely block the renal nerve activities. Renal denervation reduces or completely blocks renal sympathetic nerve activity, decreases sodium retention and renin release as well as reduces renal vascular resistance, and improves renal blood flow and glomerular filtration rate.

The objective of renal denervation is to neutralize the effect of renal sympathetic system which is involved in arterial hypertension. Device-based renal denervation may achieve such objective, but may produce possible complications of renal artery/vein stenosis. Thus, there is a need for a device that can perform renal denervation with reduced risk of renal artery/vein stenosis.

U.S. Patent Application Publication No. 2011/0118726 discloses a single basket of multiple spines with electrodes arranged on the spines in a staggered configuration. It is incorporated herein by reference in its entirety. This reference does not disclose a flexible joint between longitudinally spaced, neighboring baskets/sets of electrodes.

### BRIEF SUMMARY OF THE INVENTION

Embodiments of the present invention are directed to multiple staggered ablation elements that are longitudinally spaced and connected via flexible joints. In general, multiple baskets/sets of spines are spaced longitudinally and connected longitudinally via flexible joints, and the baskets/sets of spines have electrodes that are arranged in a staggered configuration. The staggered ablation elements or electrodes which are energized to produce ablation zones that span one or more open arc segments around the longitudinal axis, and the ablation zones of all the ablation elements projected longitudinally onto any lateral plane which is perpendicular to the longitudinal axis span a substantially closed loop around the longitudinal axis. The renal nerves are oriented generally longitudinally. Because the ablation zones do not form a closed loop, the risk of renal artery/vein stenosis is reduced or eliminated. On the other hand, because the ablation zones of all the ablation elements projected longitudinally onto any lateral plane span a substantially closed loop, a substantially complete renal denervation is achieved.

In a specific embodiment, two baskets of paired electrodes are connected via a flexible joint. The paired electrodes of the two baskets are staggered such that the ablation zones of the electrodes span one or more open arc segments around the longitudinal axis of the catheter, and the ablation zones of all the electrodes projected longitudinally onto any lateral plane which is perpendicular to the longitudinal axis span a substantially closed loop around the longitudinal axis. The flexible joint allows the two baskets of paired electrodes to be oriented at an angle with respect to one another to make it easier to move them inside a patient's anatomy such as a renal artery and to facilitate better contact between the electrodes and the target ablation areas.

In accordance with an aspect of the present invention, an ablation catheter comprises: an elongated catheter body extending longitudinally between a proximal end and a distal end along a longitudinal axis; and an electrode assembly connected to the catheter body, the electrode assembly comprising a plurality of longitudinally spaced sets of spines, which include a distal set of spines each having a distal end connected to a spine distal junction and a proximal end connected to a first joint, and a proximal set of spines each having a proximal end connected to the distal end of the catheter body and a distal end connected to the first joint or a second joint. If the distal ends of the proximal set of spines are connected to a second joint, then the electrode assembly includes one or more sets of spines connected between the first joint and the second joint in a manner such that neighboring sets of spines spaced longitudinally from one another are connected by one of a plurality of joints that include the first and second joints, the plurality of joints each being flexible to permit bending along the longitudinal axis. Each spine includes an intermediate segment, a proximal stiffness change between the proximal end and the intermediate segment of the spine, and a distal stiffness change between the distal end and the intermediate segment of the spine, wherein the spines include a plurality of ablation electrodes on the intermediate segments. The electrode assembly is movable between a collapsed arrangement and an expanded arrangement with the intermediate segments of the spines in the expanded arrangement moving outwardly relative to the proximal ends and distal ends of the spines with respect to the collapsed arrangement.

In some embodiments, the joints each include a tubular member having one or more cuts generally in a circumferential direction, the one or more cuts being at least partially through a thickness of the tubular member. The one or more cuts are configured to reduce flexural rigidity of the joints to allow bending with substantially no reduction of longitudinal rigidity of the joints against longitudinal tension and compression. The joints each include a tubular member having multiple longitudinally spaced rows of cuts generally in a circumferential direction, the one or more cuts being at least partially through a thickness of the tubular member. The joints each comprise a coil having multiple turns around the longitudinal axis. The length of each spine is approximately equal to a length of a circumference of a circular cylinder around the longitudinal axis on which the spines are disposed in the collapsed position. Each ablation electrode is to be energized to produce an ablation zone. The ablation electrodes are distributed on the intermediate segments in a staggered configuration such that the ablation zones of the ablation electrodes span one or more open arc segments around the longitudinal axis, and the ablation zones of all the ablation electrodes projected longitudinally onto any lateral plane which is perpendicular to the longitudinal axis span a substantially closed loop around the longitudinal axis.

In specific embodiments, the ablation electrodes in the expanded arrangement contact surfaces to be ablated. The ablation electrodes in the expanded arrangement span one or more open arc segments around the longitudinal axis, and all the ablation electrodes in the expanded arrangement projected longitudinally onto any lateral plane which is perpendicular to the longitudinal axis span a substantially closed loop around the longitudinal axis. At least some of the ablation electrodes each have a lateral dimension along a circumferential direction around the longitudinal axis which is greater than a longitudinal dimension along the longitudinal direction. The ablation catheter further comprises a tubing coupled with the electrode assembly; and a mechanism disposed near the proximal end of the catheter body and coupled with the tubing to manipulate the tubing to move the electrode assembly between the collapsed arrangement and the expanded arrangement. The tubing is coupled with the spine distal junction of the electrode assembly, to pull the spine distal junction in the proximal direction to move the electrode assembly to the expanded arrangement and to push the spine distal junction in the distal direction to move the electrode assembly to the collapsed arrangement. The tubing is flexible in bending along the longitudinal axis.

In accordance with another aspect of the invention, an ablation catheter comprises: an elongated catheter body extending longitudinally between a proximal end and a distal end along a longitudinal axis; and an ablation element assembly connected to the catheter body, the ablation element assembly comprising a plurality of longitudinally spaced sets of spines, which include a distal set of spines each having a distal end connected to a spine distal junction and a proximal end connected to a first joint, and a proximal set of spines each having a proximal end connected to the distal end of the catheter body and a distal end connected to the first joint or a second joint. If the distal ends of the proximal set of spines are connected to a second joint, then the ablation element assembly includes one or more sets of spines connected between the first joint and the second joint in a manner such that neighboring sets of spines spaced longitudinally from one another are connected by one of a plurality of joints that include the first and second joints, the plurality of joints each being flexible to permit bending along the longitudinal axis. Each spine includes an intermediate segment, a proximal stiffness change between the proximal end and the intermediate segment of the spine, and a distal stiffness change between the distal end and the intermediate segment of the spine, wherein the spines include a plurality of ablation elements on the intermediate segments. The ablation element assembly is movable between a collapsed arrangement and an expanded arrangement with the intermediate segments of the spines in the expanded arrangement moving outwardly relative to the proximal ends and distal ends of the spines with respect to the collapsed arrangement.

In some embodiments, the joints each include a tubular member having one or more cuts generally in a circumferential direction, the one or more cuts being at least partially through a thickness of the tubular member. The joints each comprise a coil having multiple turns around the longitudinal axis. The length of each spine is approximately equal to a length of a circumference of a circular cylinder around the longitudinal axis on which the spines are disposed in the collapsed position. Each ablation element is to be energized to produce an ablation zone. The ablation elements are distributed on the intermediate segments in a staggered configuration such that the ablation zones of the ablation elements span one or more open arc segments around the longitudinal axis, and the ablation zones of all the ablation elements projected longitudinally onto any lateral plane which is perpendicular to the longitudinal axis span a substantially closed loop around the longitudinal axis. The ablation elements in the expanded arrangement contact surfaces to be ablated. The ablation elements in the expanded arrangement span one or more open arc segments around the longitudinal axis, and all the ablation elements in the expanded arrangement projected longitudinally onto any lateral plane which is perpendicular to the longitudinal axis span a substantially closed loop around the longitudinal axis. The ablation catheter further comprises a tubing coupled with the ablation element assembly; and a mechanism disposed near the proximal end of the catheter body and coupled with the tubing to manipulate the tubing to move the ablation element assembly between the collapsed arrangement and the expanded arrangement. The tubing is flexible in bending along the longitudinal axis.

These and other features and advantages of the present invention will become apparent to those of ordinary skill in the art in view of the following detailed description of the specific embodiments.

### BRIEF DESCRIPTION OF THE DRAWINGS

FIG. 1 illustrates an assembly of staggered ablation elements for a catheter having two baskets of paired electrodes connected via a flexible joint in an expanded position according to an embodiment of the present invention.

FIG. 2 shows the assembly of FIG. 1 in a collapsed position.

FIG. 3 shows the assembly of FIG. 1 in a collapsed position with bending of the flexible joint.

FIG. 4 shows the assembly of FIG. 1 in an expanded position with bending of the flexible joint.

FIG. 5 shows an example of a spine employing hinges according to another configuration.

FIG. 6 is a cross-sectional view of a spine illustrating an example of a temperature sensor and an irrigation fluid channel.

FIG. 7 illustrates the ablation zones of the ablation elements that span open arc segments around the longitudinal axis of the catheter.

FIG. 8 illustrates the ablation zones of all the ablation elements that, when projected longitudinally onto any lateral plane which is perpendicular to the longitudinal axis, span a substantially closed loop around the longitudinal axis of the catheter.

FIG. 9 illustrates an over-the-wire configuration for introducing the assembly of staggered ablation elements on a catheter to the treatment site by passing the guide wire through an internal lumen of the catheter.

FIG. 10 illustrates another configuration of an assembly of staggered ablation elements on a catheter.

FIG. 11 illustrates another configuration of an assembly of staggered ablation elements on a catheter.

FIG. 12 shows an example of a mechanism for manipulating the movement of the spines of the ablation electrode assembly.

FIG. 13 shows a cross-sectional view of the catheter of FIG. 12 between the handle and the ablation electrode assembly.

### DETAILED DESCRIPTION OF THE INVENTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment,” “this embodiment,” or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Exemplary embodiments of the invention, as will be described in greater detail below, provide multiple staggered ablation elements such as electrodes that are longitudinally spaced and connected via flexible joints.

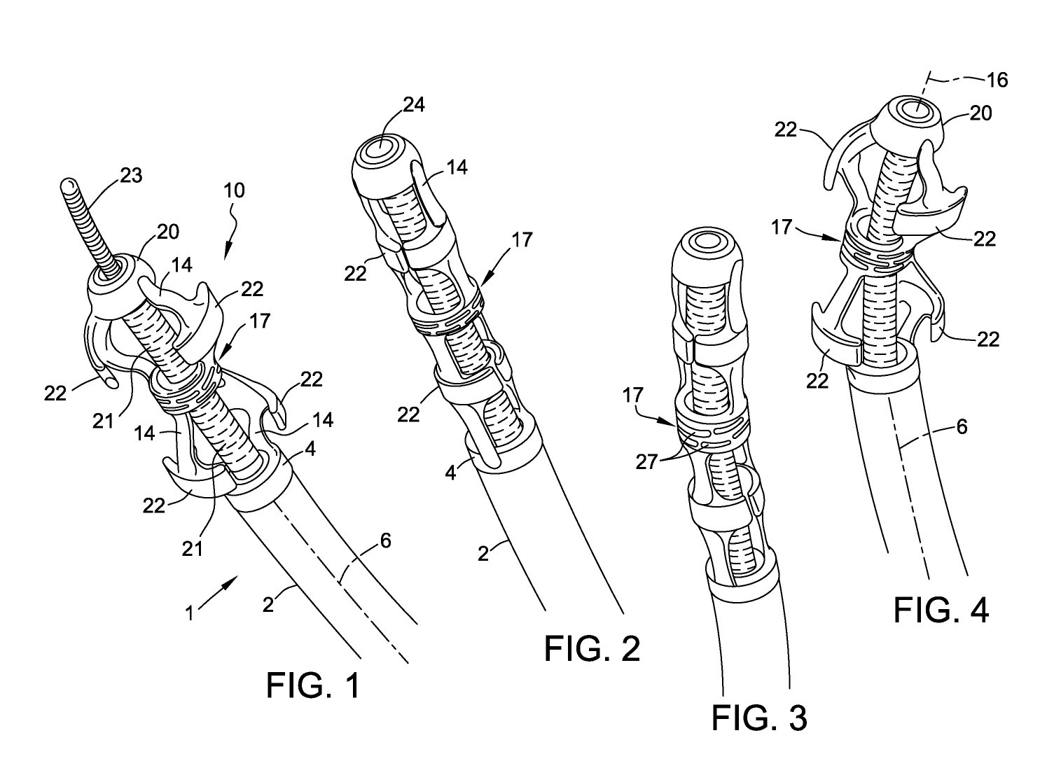

FIG. 1 illustrates an assembly of staggered ablation elements for a catheter having two baskets of paired electrodes connected via a flexible joint in an expanded position according to an embodiment of the present invention. In general, the assembly has multiple baskets and each basket has a set of spines supporting a plurality of electrodes. In the perspective view of FIG. 1, an ablation catheter 1 includes an elongated catheter body 2 extending longitudinally between a proximal end (not shown) and a distal end 4 along a longitudinal axis 6. An ablation element assembly 10 includes a plurality of ablation elements 22 connected to the catheter body 2. The ablation elements 22 are discretely spaced from each other longitudinally and/or laterally, and at least two of the ablation elements 22 are spaced from one another longitudinally.

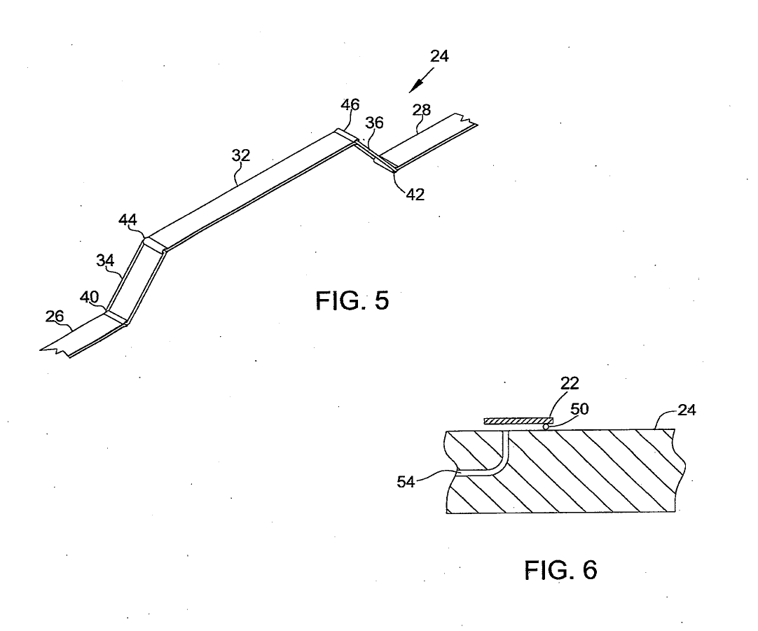

In this embodiment, the ablation elements 22 are electrodes such as RF electrodes. The ablation electrode assembly 10 is connected to the distal end 4 of the catheter body 2. As seen in FIGS. 1-4, the electrode assembly 10 has two baskets of paired electrodes 22 that are provided on spines 14, which may be oriented generally longitudinally. For the distal basket, each spine 14 has a proximal end connected to a flexible joint 17 and a distal end connected to a spine distal junction 20. For the proximal basket, each spine 14 has a proximal end connected to the distal end 4 of the catheter body 2 and a distal end connected to the flexible joint 17. Each spine 14 includes an intermediate segment on which the electrode 22 is provided, a proximal stiffness change between the proximal end of the spine 14 and the intermediate segment of the spine 14, and a distal stiffness change between the distal end of the spine 14 and the intermediate segment of the spine 14. The stiffness change may be produced by a change in size or shape or the use of a hinge (see FIG. 5). In the embodiment shown, the electrodes 22 are arranged in a staggered configuration having multiple baskets/sets of spines 14 of staggered electrodes 22, wherein each pair of longitudinally neighboring baskets/sets of spines 14 are separated by a flexible joint 17. Each spine 14 has an electrode 22 in this embodiment. In FIG. 1, each basket has two spines 14 spaced 180° apart and the two baskets are staggered by 90° to provide evenly spaced staggered spines.

FIG. 2 shows the assembly 10 of FIG. 1 in a collapsed position. The electrode assembly 10 is movable between a collapsed arrangement (FIG. 2) and an expanded arrangement (FIG. 1) with the intermediate segments of the spines 14 in the expanded arrangement moving outwardly relative to the proximal ends and distal ends of the spines 14 with respect to the collapsed arrangement (FIG. 2). The movement between the collapsed position and the expanded position may be achieved by pulling and pushing/releasing a flexible tube 21 serving as an activation member that is attached to the spine distal junction 20, with respect to the distal end 4 of the catheter body 2. The flexible tube 21 extends through the interior of the catheter body 2. It is sufficiently flexible so as to bend with the assembly 10 as the assembly bends at the flexible joint(s) 17 but is sufficiently rigid to act as an activation member for moving the baskets of the assembly 10 between the collapsed position and the expanded position. The flexible tube 21 preferably has a lumen 24 provided to accommodate a guide wire 23.

FIG. 3 shows the assembly 10 of FIG. 1 in a collapsed position with bending of the flexible joint 17. FIG. 4 shows the assembly 10 of FIG. 1 in an expanded position with bending of the flexible joint 17. In the embodiment shown, the rigidity of the flexible joint 17 is weakened by cuts or grooves or slots 27 along the circumference so that the assembly 10 can bend to a curved position at the flexible joint 17 along the longitudinal axis. The cuts 27 may be through thickness or not, and may be a mechanical cut, a laser cut, or the like. In this embodiment, there are two to three rows of cuts 27 which are staggered, and each row has a plurality of circumferential cuts which are circumferentially spaced apart. One can design the number, distribution, size, and shape of the cuts 27 to achieve the desirable bending of the flexible joint 17. Preferably, the one or more cuts are configured to reduce flexural rigidity of the joints to allow bending (e.g., up to about 45 degrees) with substantially no reduction of longitudinal rigidity of the joints against longitudinal tension and compression (e.g., permissible tensile or compressive deflection of less than about 30% in each joint in the longitudinal direction). In specific embodiments, the flexible joint 17 has sufficiently tensile strength and rigidity such that, even with the cuts 27, it can bend but it substantially does not stretch or compress in the longitudinal direction. While FIGS. 1-4 show two baskets/sets of paired spines supporting electrodes, three or more baskets/sets may be used in other embodiments and three or more spines may be provided in a basket.

As compared to the assemblies shown in US2011/0118726, the embodiment of FIGS. 1-4 has a number of distinct characteristics. The use of multiple baskets/sets connected by flexible joints allows the electrode assembly 10 to bend and conform to the patient's arteries or other interior vessels or cavities, making it easier to move the electrode assembly 10 through such cavities and go deeper into such cavities. In addition, by bending the assembly 10, the electrodes 22 can make better contact or at least more predictable contact with tissue to achieve improved ablation and/or lesion formation. This is further enhanced by using shorter spines 14 per each basket in the multiple basket configuration of FIGS. 1-4 as compared to the longer spines in the single basket configuration disclosed in US2011/0118726. In specific embodiments, the length of each spine 14 in a basket is comparable to (typically approximately equal to or slightly greater, e.g., within about 10%) the length of the circumference of a circular cylinder (around the longitudinal axis) on which the spines 14 are disposed in the collapsed position. The multiple basket configuration of FIGS. 1-4 generally means the electrode array can have a much lower profile than that of the single basket configuration with the same number of electrodes space longitudinally. This feature can render the multiple basket configuration easier to maneuver and more widely applicable. Therefore, the multiple basket configuration provides the capability to engage and ablate in torturous vessels through improved contact with vessel wall. In addition to access via the femoral artery or the like, the apparatus also lends itself to entry by radial access into the patient.

In use, the catheter 1 with the electrode assembly 10 is inserted into a blood vessel or the like in the collapsed arrangement (FIG. 2 or 3) (inside a guiding sheath or the like) and deployed into the expanded arrangement (FIG. 1 or 4). To allow blood flow in the blood vessel across the electrode assembly 10 and reduce or avoid obstruction, the spines 14 each have narrow intermediate segment, proximal leg, and distal leg. In FIGS. 1-4, the intermediate segment is wider while the proximal leg and distal leg are tapered so as to be smaller in cross-section than the intermediate segment, thereby reducing obstruction. Furthermore, the electrode assembly 10 preferably has no sharp corners or edges but has rounded corners and edges to facilitate easier and smoother movement within the blood vessel.

FIG. 5 shows an example of a spine employing hinges according to another configuration. Each spine 14 has a proximal end 26 connected to the catheter body 2 or a flexible joint and a distal end 28 connected to a flexible joint or the spine distal junction 20. Each spine 14 includes an intermediate segment 32, a proximal stiffness change between the proximal end 26 and the intermediate segment 32 of the spine 14, and a distal stiffness change between the distal end 28 and the intermediate segment 32 of the spine 14. The spine 14 includes at least one ablation electrode (such as electrode 22 of FIG. 1) on the intermediate segment 32. Each spine 14 includes a proximal leg 34 coupled between the intermediate segment 32 and the proximal end 26 of the spine 14, and a distal leg 36 coupled between the intermediate segment 32 and the distal end 28 of the spine 14. Each spine 14 includes a proximal hinge 44 coupled between the proximal leg 34 and the intermediate segment 32 and a distal hinge 46 coupled between the distal leg 36 and the intermediate segment 32. The hinges 44, 46 represent the stiffness changes in this embodiment to facilitate movement of the intermediate segment 32 of the spine 14 between the collapsed arrangement and the expanded arrangement. In addition, each spine 14 may further include a proximal end hinge 40 coupled between the proximal leg 34 and the proximal end 26 and a distal end hinge 42 coupled between the distal leg 36 and the distal end 28 to further facilitate movement of the intermediate segment 32 of the spine 14 between the collapsed arrangement and the expanded arrangement. Tapered/rounded corners may replace sharp corners to facilitate easier and smoother movement of the electrode assembly within the blood vessel. In one example, any or all of the proximal leg 34, the distal leg 36, the proximal end hinge 40, and the distal end hinge 42 of the spine 14 may be resiliently biased (e.g., with a spring or a memory material) to move the electrode assembly 10 toward the expanded arrangement (FIG. 1 or 4) in some embodiments and toward the collapsed arrangement (FIG. 2 or 3) in other embodiments.

In a specific embodiment, the spines are preformed in the expanded position and are collapsed within a guiding sheath prior to introducing the catheter into the patient. After the spines are placed in the desired location within the patient, the guiding sheath is pulled back to allow the spines to move to the expanded position to which they are resiliently biased.

The ablation electrodes 22 in the expanded arrangement (FIG. 1 or 4) contact surfaces to be ablated to ablate tissue and/or denervate nerves. To ensure surface contact for the ablation electrodes 22, the intermediate segments preferably have sufficient stiffness to avoid or minimize bending in the expanded arrangement. The electrode assembly 10 moves from the collapsed arrangement to the expanded arrangement by any suitable mechanism. In one example, any or all of the proximal leg and the distal leg of the spine 14 may be resiliently biased (e.g., with a spring or a memory material) to move the electrode assembly 10 toward the expanded arrangement. The flexible tube 21 can be used to push the spine distal junction 20 away from the distal end 4 to move the assembly 10 against the biasing forces to the collapsed arrangement. In another example, the flexible tube 21 can be used to pull the spine distal junction 20 toward the distal end 4 to move the assembly 10 to the expanded arrangement, and can be released or used to push the spine distal junction 20 away from the distal end 4 to move the electrode assembly 10 toward the collapsed arrangement.

A plurality of temperature sensors 50 are thermally coupled with the plurality of ablation electrodes 22 to measure temperatures of the ablation electrodes.

FIG. 6 is a cross-sectional view of the spine 14 illustrating an example of a temperature sensor 50 disposed adjacent the electrode 22 supported on the spine 14. In addition, the spines 14 may include a plurality of irrigation fluid channels 54 near the plurality of ablation electrodes 22 to direct irrigation fluid toward the ablation electrodes 22, as seen in FIG. 6.

In FIGS. 1-4, the ablation electrodes 22 each have a lateral dimension (along the circumferential direction) which is greater than a longitudinal dimension (along the longitudinal direction) thereof and greater than the lateral dimension of the spine 14 that supports the electrode 22. Each electrode 22 has the shape of a circumferential arc that produces an ablation zone that is oriented laterally with respect to the longitudinal axis. Such an ablation zone is more efficient and effective for denervating renal nerves that are oriented generally longitudinally. Of course, other shapes and sizes for the electrodes 22 may be used in other embodiments.

The spines 14 are configured to facilitate movement of the electrode assembly 10 from the collapsed arrangement to the expanded arrangement. For example, the proximal leg has a lower stiffness than the intermediate segment and the distal leg has a lower stiffness than the intermediate segment. As a result, the proximal leg and the distal leg will bend or deform under a force that moves the electrode assembly 10 to the expanded arrangement. That force may be produced by at least one of the spines 14 made of a shape memory material (e.g., Nitinol) and/or by the flexible tube 21 serving as an activation member.

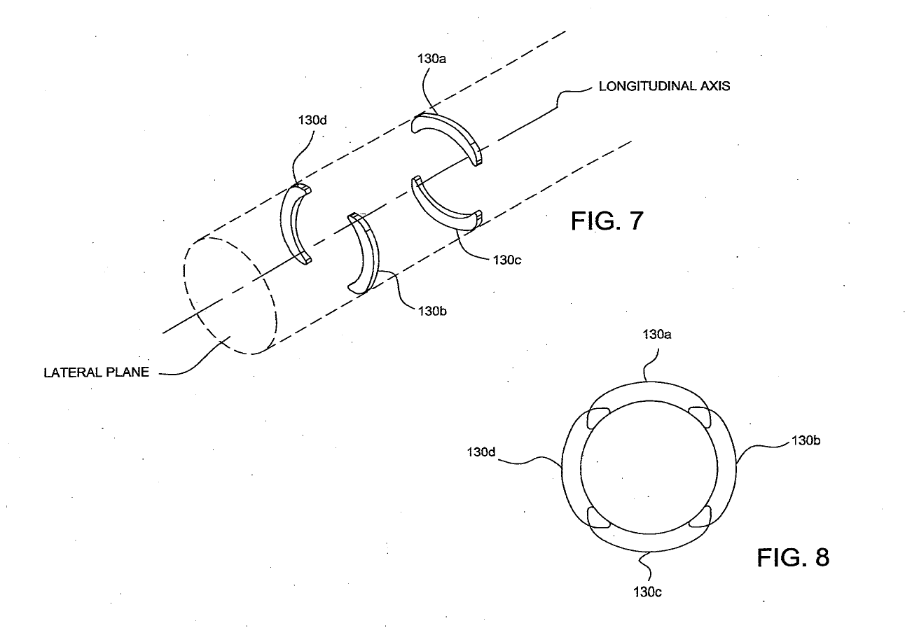

FIG. 7 illustrates the ablation zones 130 of the ablation elements that span open arc segments around the longitudinal axis of the catheter. Each ablation element has a corresponding ablation zone (130a, 130b, 130c, . . . ). For each ablation element, the ablation zone is a region that is energized with sufficient energy to ablate tissue (e.g., tissue reaching a temperature of at least about 45-50° C. for several seconds) or denervate nerves (e.g., tissue immediately around nerves reaching a temperature of at least about 45-50° C. for several seconds) within the ablation zone. The ablation zones 130 may be about the same in shape and size as the corresponding ablation elements. For RF electrodes or the like, the ablation zones are likely to be larger than the corresponding RF electrodes. The ablation elements are distributed in a staggered configuration such that the ablation zones 130 of the ablation elements span one or more open arc segments around the longitudinal axis.

FIG. 8 illustrates the ablation zones 130 of all the ablation elements that, when projected longitudinally onto any lateral plane which is perpendicular to the longitudinal axis, span a closed loop around the longitudinal axis of the catheter. In the embodiment illustrated by FIG. 8, the closed loop is completely closed. In other embodiments, the loop is substantially closed. The substantially closed loop has one or more open portions. The aggregate open portion of the substantially closed loop is about 30 percent or less of the substantially closed loop. An energy source supplies energy to the independently controlled ablation elements simultaneously or sequentially or in an arbitrary order to produce the ablation zones. In this way, tissue ablation or renal denervation or the like can be performed efficiently, effectively, and quickly, and in accordance with user selection.

In specific embodiments, the ablation electrodes in the expanded arrangement span one or more open arc segments around the longitudinal axis, and all the ablation electrodes in the expanded arrangement projected longitudinally onto any lateral plane which is perpendicular to the longitudinal axis span a substantially closed loop around the longitudinal axis. The substantially closed loop has one or more open portions. The aggregate open portion of the substantially closed loop is about 30 percent or less of the substantially closed loop.

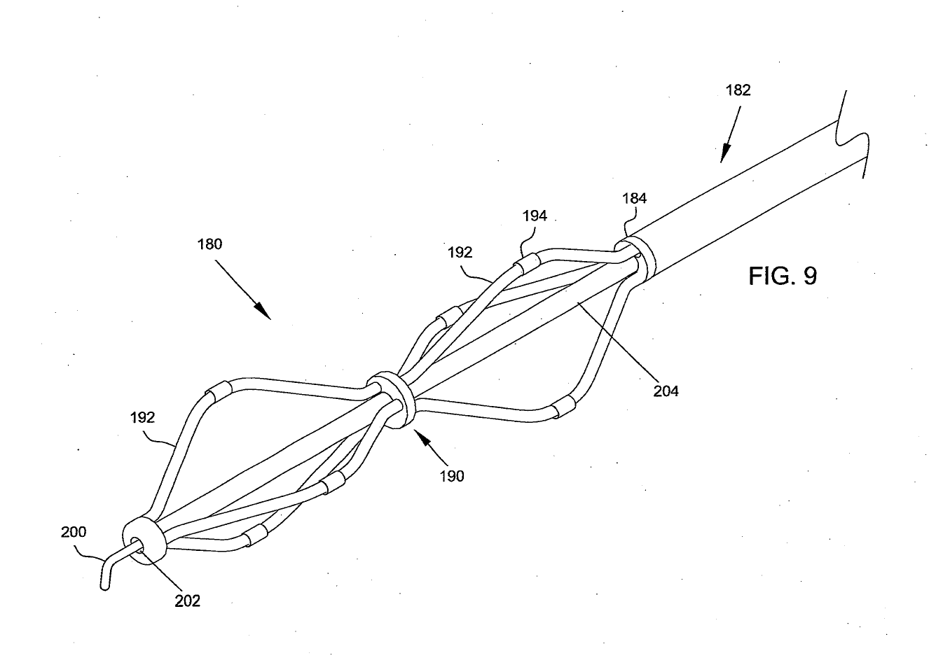

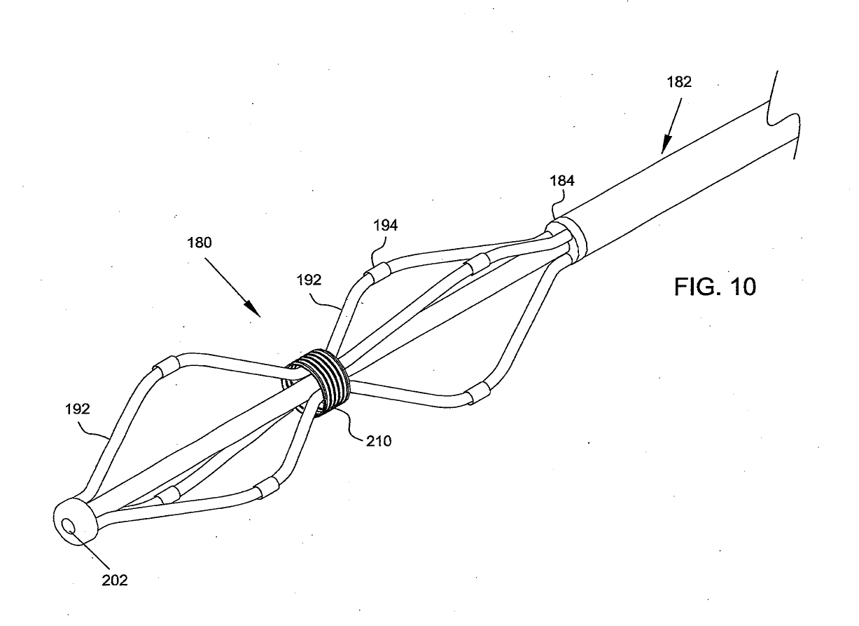

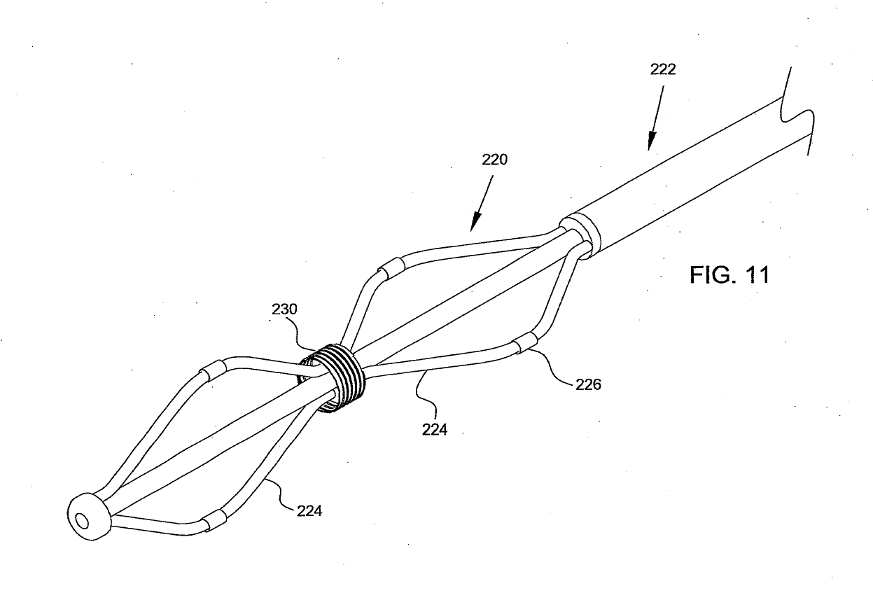

FIG. 9 illustrates an over-the-wire configuration for introducing an assembly 180 of staggered ablation elements on a catheter 182 to the treatment site by passing a guide wire 200 through an internal lumen of the catheter 182 (see guidewire 23 in FIG. 1). The guide wire 200 extends through an opening 202 at the distal end of the assembly 180, and through a tube 204 that extends through the assembly 180 to the internal lumen of the catheter 182 from its distal end 184 to its proximal end. The distal end of the assembly 180 is disposed distally of the distal end 184 of the catheter 182. The assembly 180 in FIG. 9 includes two baskets connected via a flexible joint 190. The flexible joint 190 is a tubular member having cuts (not shown) similar to those illustrated in FIGS. 1-4, namely, multiple longitudinally spaced rows of cuts generally in a circumferential direction. Each basket includes three spines 192 each having an electrode 194. The spines 192 are spaced 120° apart and the spines 192 of the two baskets are staggered by 60° to provide evenly spaced staggered spines.

FIG. 10 illustrates another configuration of an assembly of staggered ablation elements on a catheter. This configuration of the assembly 180 and catheter 182 is similar to that of FIG. 9, except the tubular joint 190 with cuts is replaced by a coil joint 210. The coil joint 210 includes multiple turns around the longitudinal axis and may be constructed in a manner similar to a helical spring. The spines 192 may be welded to the coil joint 210 or the spines 192 and the coil joint 210 may be laser cut all in one piece.

FIG. 11 illustrates another configuration of an assembly of staggered ablation elements on a catheter. This configuration of the assembly 220 and catheter 222 is similar to that of FIG. 10, except each set of spines has two spines 224 spaced 180° apart (similar to FIGS. 1-4). Each spine 224 has an electrode 226. The two sets of spines 224 are connected via a coil joint 230.

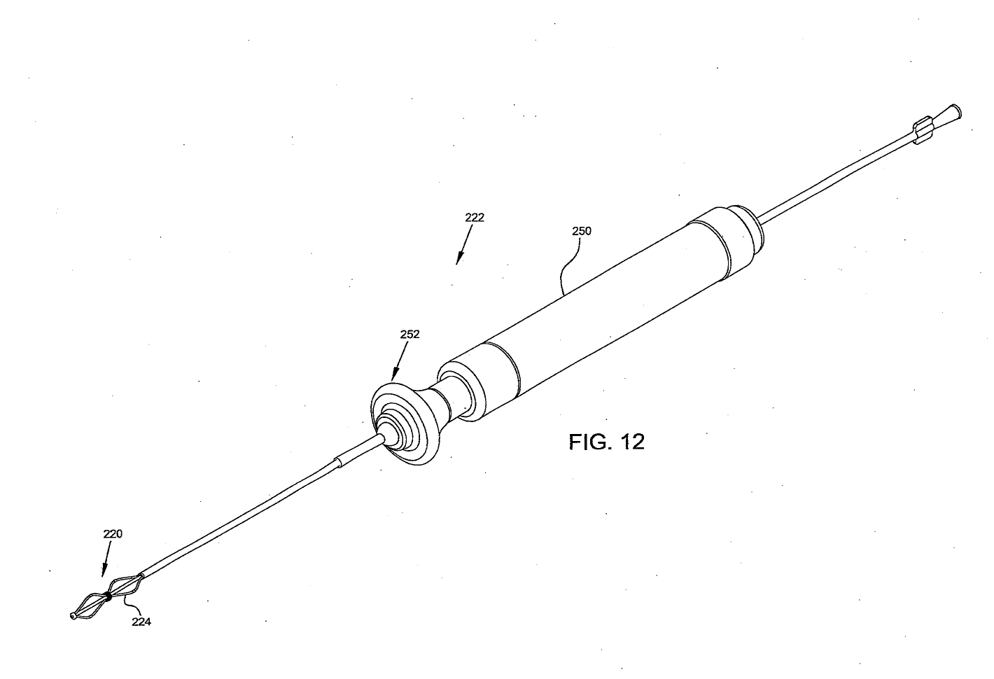

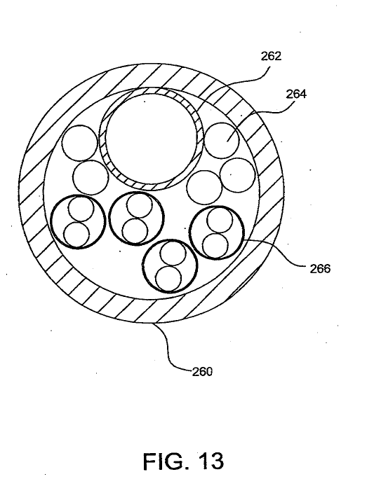

FIG. 12 shows an example of a mechanism for manipulating the movement of the spines of the ablation electrode assembly. FIG. 12 shows a proximal handle 250 at or near the proximal end of the catheter 222 of FIG. 11. A push-pull mechanism 252 is provided at the handle 250 and can be pushed or pulled by the user. The user can use the push-pull mechanism 252 to push/pull a push-pull tubing (21 in FIG. 1-4 or 262 in FIG. 13) to move the spines 224 between the expanded configuration/position and the collapsed configuration/position.

FIG. 13 shows a cross-sectional view of the catheter 222 of FIG. 12 between the handle 250 and the ablation electrode assembly 220 having the spines 224. Disposed inside the catheter body are a pull tubing 262, conductor wires 264 for supplying energy to the electrodes, and thermal sensors 266. The pull tubing 262 is preferably flexible in bending to bend with the ablation electrode assembly at the flexible joint(s). In the embodiment shown, the distal end of the pull tubing 262 is connected to the spine distal junction (e.g., 20 in FIGS. 1-4). Pulling the pull tubing 262 in the proximal direction causes the spine distal junction 20 to move toward the distal end (e.g., 4 in FIG. 1-4) of the catheter body, thereby moving the electrode assembly to the expanded position. Pushing or releasing the pull tubing 262 in the distal direction causes the spine distal junction 20 to move away from the distal end 4 of the catheter body, thereby moving the electrode assembly to the collapsed position.

In the description, numerous details are set forth for purposes of explanation in order to provide a thorough understanding of the present invention. However, it will be apparent to one skilled in the art that not all of these specific details are required in order to practice the present invention. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. An ablation catheter comprising:
an elongated catheter body extending longitudinally between a proximal end and a distal end along a longitudinal axis; and
an electrode assembly connected to the catheter body, the electrode assembly comprising a plurality of longitudinally spaced sets of spines, which include a distal set of spines each having a distal end connected to a spine distal junction and a proximal end connected to a first joint, and a proximal set of spines each having a proximal end connected to the distal end of the catheter body and a distal end connected to the first joint or a second joint;
wherein if the distal ends of the proximal set of spines are connected to a second joint, then the electrode assembly includes one or more sets of spines connected between the first joint and the second joint in a manner such that neighboring sets of spines spaced longitudinally from one another are connected by one of a plurality of joints that include the first and second joints, the plurality of joints each being flexible to permit bending along the longitudinal axis;
wherein each spine includes an intermediate segment, a proximal stiffness change between the proximal end and the intermediate segment of the spine, and a distal stiffness change between the distal end and the intermediate segment of the spine, wherein the spines include a plurality of ablation electrodes on the intermediate segments; and
wherein the electrode assembly is movable between a collapsed arrangement and an expanded arrangement with the intermediate segments of the spines in the expanded arrangement moving outwardly relative to the proximal ends and distal ends of the spines with respect to the collapsed arrangement.

2. The ablation catheter of claim 1,
wherein the joints each include a tubular member having one or more cuts generally in a circumferential direction, the one or more cuts being at least partially through a thickness of the tubular member.

3. The ablation catheter of claim 2,
wherein the one or more cuts are configured to reduce flexural rigidity of the joints to allow bending with substantially no reduction of longitudinal rigidity of the joints against longitudinal tension and compression.

4. The ablation catheter of claim 1,
wherein the joints each include a tubular member having multiple longitudinally spaced rows of cuts generally in a circumferential direction, the one or more cuts being at least partially through a thickness of the tubular member.

5. The ablation catheter of claim 1,
wherein the joints each comprise a coil having multiple turns around the longitudinal axis.

6. The ablation catheter of claim 1,
wherein the length of each spine is approximately equal to a length of a circumference of a circular cylinder around the longitudinal axis on which the spines are disposed in the collapsed position.

7. The ablation catheter of claim 1,
wherein each ablation electrode is to be energized to produce an ablation zone;
wherein the ablation electrodes are distributed on the intermediate segments in a staggered configuration such that the ablation zones of the ablation electrodes span one or more open arc segments around the longitudinal axis, and the ablation zones of all the ablation electrodes projected longitudinally onto any lateral plane which is perpendicular to the longitudinal axis span a substantially closed loop around the longitudinal axis.

8. The ablation catheter of claim 1,
wherein the ablation electrodes in the expanded arrangement contact surfaces to be ablated; and
wherein the ablation electrodes in the expanded arrangement span one or more open arc segments around the longitudinal axis, and all the ablation electrodes in the expanded arrangement projected longitudinally onto any lateral plane which is perpendicular to the longitudinal axis span a substantially closed loop around the longitudinal axis.

9. The ablation catheter of claim 1,
wherein at least some of the ablation electrodes each have a lateral dimension along a circumferential direction around the longitudinal axis which is greater than a longitudinal dimension along the longitudinal direction.

10. The ablation catheter of claim 1, further comprising:
a tubing coupled with the electrode assembly; and
a mechanism disposed near the proximal end of the catheter body and coupled with the tubing to manipulate the tubing to move the electrode assembly between the collapsed arrangement and the expanded arrangement.

11. The ablation catheter of claim 10,
wherein the tubing is coupled with the spine distal junction of the electrode assembly, to pull the spine distal junction in the proximal direction to move the electrode assembly to the expanded arrangement and to push the spine distal junction in the distal direction to move the electrode assembly to the collapsed arrangement.

12. The ablation catheter of claim 10,
wherein the tubing is flexible in bending along the longitudinal axis.

13. An ablation catheter comprising:
an elongated catheter body extending longitudinally between a proximal end and a distal end along a longitudinal axis; and
an ablation element assembly connected to the catheter body, the ablation element assembly comprising a plurality of longitudinally spaced sets of spines, which include a distal set of spines each having a distal end connected to a spine distal junction and a proximal end connected to a first joint, and a proximal set of spines each having a proximal end connected to the distal end of the catheter body and a distal end connected to the first joint or a second joint;
wherein if the distal ends of the proximal set of spines are connected to a second joint, then the ablation element assembly includes one or more sets of spines connected between the first joint and the second joint in a manner such that neighboring sets of spines spaced longitudinally from one another are connected by one of a plurality of joints that include the first and second joints, the plurality of joints each being flexible to permit bending along the longitudinal axis;
wherein each spine includes an intermediate segment, a proximal stiffness change between the proximal end and the intermediate segment of the spine, and a distal stiffness change between the distal end and the intermediate segment of the spine, wherein the spines include a plurality of ablation elements on the intermediate segments; and
wherein the ablation element assembly is movable between a collapsed arrangement and an expanded arrangement with the intermediate segments of the spines in the expanded arrangement moving outwardly relative to the proximal ends and distal ends of the spines with respect to the collapsed arrangement.

14. The ablation catheter of claim 13,
wherein the joints each include a tubular member having one or more cuts generally in a circumferential direction, the one or more cuts being at least partially through a thickness of the tubular member.

15. The ablation catheter of claim 13,
wherein the joints each comprise a coil having multiple turns around the longitudinal axis.

16. The ablation catheter of claim 13,
wherein the length of each spine is approximately equal to a length of a circumference of a circular cylinder around the longitudinal axis on which the spines are disposed in the collapsed position.

17. The ablation catheter of claim 13,
wherein each ablation element is to be energized to produce an ablation zone;
wherein the ablation elements are distributed on the intermediate segments in a staggered configuration such that the ablation zones of the ablation elements span one or more open arc segments around the longitudinal axis, and the ablation zones of all the ablation elements projected longitudinally onto any lateral plane which is perpendicular to the longitudinal axis span a substantially closed loop around the longitudinal axis.

18. The ablation catheter of claim 13,
wherein the ablation elements in the expanded arrangement contact surfaces to be ablated; and
wherein the ablation elements in the expanded arrangement span one or more open arc segments around the longitudinal axis, and all the ablation elements in the expanded arrangement projected longitudinally onto any lateral plane which is perpendicular to the longitudinal axis span a substantially closed loop around the longitudinal axis.

19. The ablation catheter of claim 13, further comprising:
a tubing coupled with the ablation element assembly; and
a mechanism disposed near the proximal end of the catheter body and coupled with the tubing to manipulate the tubing to move the ablation element assembly between the collapsed arrangement and the expanded arrangement.

20. The ablation catheter of claim 19,
wherein the tubing is flexible in bending along the longitudinal axis.
